(19) United States
(12) Patent Application Publication
(10) Pub. No.: US 2026/0345077 A1
(43) Pub. Date: Nov. 5, 2026

(54) SMART WATCH AND PHYSIOLOGICAL DATA MEASUREMENT METHOD CROSS-REFFERENCE TO THE RELATED APPLICATIONS

(71) Applicant: GOERTEK TECHNOLOGY CO., LTD, Qingdao, Shandong (CN)

(72) Inventors: Yong QIE, Qingdao, Shandong (CN); Jinshan ZHENG, Qingdao, Shandong (CN); Haisong LIANG, Qingdao, Shandong (CN); Jianlei REN, Qingdao, Shandong (CN); Huan LI, Qingdao, Shandong (CN); Yulong SHI, Qingdao, Shandong (CN)

(21) Appl. No.: 19/116,404

(22) PCT Filed: Oct. 24, 2023
(86) PCT No.: PCT/CN2023/126160

§ 371 Date: Mar. 28, 2025

(30) Foreign Application Priority Data
Oct. 31, 2022 (CN) 202211351571.0
Jul. 11, 2023 (CN) 202310847358.7

(51) Int. Cl. A61B 5/00; A61B 5/0205; A61B 5/021; A61B 5/024; A61B 5/256; A61B 5/282; A61B 5/332; A61B 7/04; G04G 21/02

(52) U.S. Cl. CPC A61B 5/681; A61B 5/0205; A61B 5/7278; G04G 21/025; A61B 5/02108; A61B 5/02416; A61B 5/02438; A61B 5/256; A61B 5/282; A61B 5/332; A61B 7/04

(57) ABSTRACT

The present disclosure provides a smart watch and a physiological data measurement method. The smart watch includes: a physiological sound signal collector configured to collect a physiological sound signal; an electrocardiogram signal collector configured to collect an electrocardiogram signal; a pulse signal collector configured to collect a pulse signal; and a control chip configured to determine physiological data, such as blood pressure data and cardiopulmonary disease data, of a user of the smart watch according to the physiological sound signal, the electrocardiogram signal and the pulse signal. The smart watch according to the present disclosure can comprehensively and accurately measure physiological data of a user of the smart watch, and then accurately monitor user's health data.

### CROSS-REFERENCE TO THE RELATED APPLICATIONS

This application claims priority to Chinese Patent Application No. 202211351571.0, entitled “Smart Watch and Blood Pressure Measurement Method,” filed with China Patent Office on Oct. 31, 2022, and Chinese Patent Application No. 202310847358.7, entitled “Smart Watch and Physiological Data Measurement Method”, filed on Jul. 11, 2023, all of which are incorporated herein by reference in their entireties.

### TECHNICAL FIELD

The present disclosure relates to the field of artificial intelligence technologies, and specifically, to a smart watch and a physiological data measurement method BACKGROUND Portable smart devices have emerged in recent years that can measure physiological data of a user, such as blood pressure.

However, the current portable smart device can generally measure only a certain type of physiological data. For example, the existing portable device for blood pressure measurement can measure the blood pressure data of the device-side user. However, the data accuracy of the user's physiological data measured by this type of portable smart device is not high.

For example, the above-mentioned portable devices for blood pressure measurement are generally divided into two types: one is to measure blood pressure by PPG (photoplethysmographic) combined with ECG (electrocardiogram) or by PPG method alone. This method needs to calibrate the devices regularly, the accuracy thereof is low, and only the research on individual blood pressure trend can be performed.

### SUMMARY

The main objective of the present disclosure is to provide a smart watch and a physiological data measurement method, which aim to perform comprehensive and accurate measurement of physiological data of a user of the smart watch, thereby accurately monitoring user health data.

To achieve the above objective, the present disclosure provides a smart watch comprising:
a physiological sound signal collector configured to collect a physiological sound signal;
an electrocardiogram signal collector configured to collect an electrocardiogram signal;
a pulse signal collector configured to collect a pulse signal; and
a control chip configured to determine physiological data of a user of the smart watch according to the physiological sound signal, the electrocardiogram signal and/or the pulse signal, wherein the physiological data comprises one or more selected from a group consisting of blood pressure data and cardiopulmonary disease data.

Optionally, the physiological sound signal collector comprises one or more selected from a group consisting of at least one bone conduction sensor, an acceleration sensor, an inertial sensor, a piezoelectric ceramic, a pressure sensor and a vibration detection sensor; and wherein the electrocardiogram signal collector comprises at least two electrocardiogram electrodes, and the pulse signal collector comprises an LED light source and a detector.

Optionally, the physiological sound collector may be installed on a body of the smart watch and/or a strap of the smart watch.

Optionally, the electrocardiogram electrode comprises at least a first electrode and a second electrode, wherein the first electrode is mounted on a lower case of a body of the smart watch, and the second electrode is mounted on an upper case or a side wall of the body of the smart watch.

Optionally, the electrocardiogram electrode further comprises a third electrode, and the third electrode is mounted on the lower case of the body of the smart watch.

Optionally, the LED light source and the detector are mounted on a lower case of a body of the smart watch.

Optionally, the smart watch further comprises:
a physiological sound signal analyzer configured to analyze and process the physiological sound signal collected by the physiological sound signal collector, and transmit the analyzed and processed physiological sound signal to the control chip;
an electrocardiogram signal analyzer configured to analyze and process the electrocardiogram signal collected by the electrocardiogram signal collector, and transmit the analyzed and processed electrocardiogram signal to the control chip;
a pulse signal analyzer configured to analyze and process the pulse signal collected by the pulse signal collector, and transmit the analyzed and processed pulse signal to the control chip.

Optionally, the smart watch further comprises: a speaker, a displayer and/or a vibration motor configured to remind the user of the smart watch to wear the smart watch on the user's wrist, so that an electrocardiogram electrode of the electrocardiogram signal collector located on a lower case of the smart watch is in close contact with the wrist of the user of the smart watch, and prompt the user of the smart watch to closely attach the worn smart watch to the user's chest while using the other hand not wearing the smart watch to lightly touch the electrocardiogram electrode of the electrocardiogram signal collector located on a top or side wall of the body of the smart watch.

Optionally, the control chip is further configured to determine whether the signal qualities of the physiological sound signal, the electrocardiogram signal and the pulse signal are greater than corresponding preset quality thresholds, so as to determine the physiological data of the user of the smart watch when each of the signal qualities is greater than the corresponding preset quality thresholds.

Optionally, to achieve the above objective, the present disclosure further provides a physiological data measurement method, including:
collecting a physiological sound signal, an electrocardiogram signal and a pulse signal of the user of the smart watch by a physiological sound signal collector, an electrocardiogram signal collector and a pulse signal collector of the smart watch, respectively; and
determining, by a control chip of the smart watch, physiological data of the user of the smart watch according to the physiological sound signal, the electrocardiogram signal and/or the pulse signal, wherein the physiological data includes one or more selected from a group consisting of blood pressure data and cardiopulmonary disease data.

Optionally, the physiological sound signal includes a heart sound signal, and the determining the physiological data of the user of the smart watch according to the physiological sound signal, the electrocardiogram signal and the pulse signal includes:
determining, by the control chip of the smart watch, the blood pressure data of the user of the smart watch according to the heart sound signal, the electrocardiogram signal, and the pulse signal.

Optionally, before the determining the blood pressure data of the user of the smart watch according to the heart sound signal, the electrocardiogram signal and the pulse signal, the method further includes:
determining whether signal qualities of the heart sound signal, the electrocardiogram signal and the pulse signal are greater than preset quality thresholds, respectively;
if the signal qualities of the heart sound signal, the electrocardiogram signal and the pulse signal are greater than the preset quality thresholds, respectively, performing the determining the blood pressure data of the user of the smart watch according to the heart sound signal, the electrocardiogram signal and the pulse signal.

Optionally, the physiological sound signal includes a heart sound/lung sound signal, the determining, by the control chip of the smart watch, the physiological data of the user of the smart watch according to the physiological sound signal includes:
determining, by a control chip of the smart watch, the cardiopulmonary disease data of the user of the smart watch according to the heart sound/lung sound signal, wherein the cardiopulmonary disease data includes one or more selected from a group consisting of heart failure, coronary heart disease, valvular heart disease, pneumonia, and crackles.

To achieve the above objective, the present disclosure further provides a terminal device, including a memory, a processor, and a blood pressure measurement program stored in the memory and executable on the processor, where the blood pressure measurement program, when executed by the processor, implements the steps of the physiological data measurement method described above.

In addition, in order to achieve the above objective, the present disclosure further provides a computer-readable storage medium having a blood pressure measurement program stored thereon, and when the blood pressure measurement program is executed by a processor, the steps of the physiological data measurement method as described above are implemented.

To achieve the above objective, the present disclosure further provides a computer program product, the computer program product includes a computer program, and when the computer program is executed by a processor, the steps of the physiological data measurement method described above are implemented.

The present disclosure provides a smart watch, a physiological data measurement method, a terminal device, a computer-readable storage medium, and a computer program product, wherein the smart watch includes: a physiological sound signal collector configured to collect a physiological sound signal; an electrocardiogram signal collector configured to collect an electrocardiogram signal; a pulse signal collector configured to collect a pulse signal; and a control chip configured to determine physiological data of a user of the smart watch according to the physiological sound signal, the electrocardiogram signal and/or the pulse signal, wherein the physiological data includes one or more selected from a group consisting of blood pressure data and cardiopulmonary disease data.

Compared with the physiological data measurement method in the related art, the smart watch in the present disclosure has a small structure size, and the measurement method is simple and easy to use. Moreover, in the present disclosure, the physiological sound signal collector, the electrocardiogram signal collector and the pulse signal collector of the smart watch simultaneously collect the physiological sound signal, the electrocardiogram signal and the pulse signal of the user of the smart watch, and then the physiological data such as the blood pressure data and the cardiopulmonary disease of the user of the smart watch is calculated by using the physiological sound signal, the electrocardiogram signal and the pulse signal through the control chip of the smart watch. Therefore, the smart watch in the present disclosure integrates various health data detection functions such as blood pressure measurement and cardiopulmonary disease detection, thereby realizing comprehensive and accurate detection of physiological data of the user of the smart watch. Moreover, the physiological sound measurement method in the present disclosure is simple and convenient to operate, and improves the user's experience when measuring physiological data.

On this basis, the present disclosure avoids the disadvantage of squeezing the wrist of the user when measuring the blood pressure through the digital cuff-type pressure monitor, and also solves the problem of inaccurate blood pressure value caused by the existing blood pressure measurement through PPG combined with ECG blood pressure measurement. The present disclosure improves the accuracy of blood pressure data measurement by performing blood pressure measurement through the physiological sound signal, the electrocardiogram signal and the pulse signal, and realizes accurate and efficient measurement of physiological data such as blood pressure.

### BRIEF DESCRIPTION OF DRAWINGS

FIG. 1 is a first schematic diagram of internal components of the smart watch according to an embodiment of the present disclosure;

FIG. 2 is a second schematic diagram of internal components of the smart watch according to an embodiment of the present disclosure;

FIG. 3-1 is a first schematic diagram of wearing the smart watch according to an embodiment of the present disclosure;

FIG. 3-2 is a second schematic diagram of wearing the smart watch according to an embodiment of the present disclosure;

FIG. 4 is a first flowchart of a physiological data measurement method according to an embodiment of the present disclosure;

FIG. 5 is a second flowchart of a physiological data measurement method according to an embodiment of the present disclosure; and

FIG. 6 is a third flowchart of a physiological data measurement method according to an embodiment of the present disclosure.

Implementations, functional features, and advantages of the present disclosure will be further described with reference to the accompanying drawings and embodiments.

### DETAILED DESCRIPTIONS

It should be understood that the specific embodiments described herein are merely used to explain the present disclosure and are not intended to limit the present disclosure.

Considering the problem that the type of physiological data measured by the existing portable smart device is excessively limited and lacks accuracy, in this embodiment, a physiological data measurement method is provided, and in an embodiment, the method is applicable to various wearable devices, including but not limited to smart watches, smart bracelets, etc.

In this embodiment, the smart watch is provided, including:
a physiological sound signal collector configured to collect a physiological sound signal;
an electrocardiogram signal collector configured to collect an electrocardiogram signal;
a pulse signal collector configured to collect a pulse signal; and
a control chip configured to determine physiological data of the user of the smart watch according to the physiological sound signal, the electrocardiogram signal and/or the pulse signal, wherein the physiological data includes one or more selected from a group consisting of blood pressure data and cardiopulmonary disease data.

It can be understood that the smart watch is a wristband device, which can be specifically divided into a body and a strap, in this embodiment, in order to be able to measure more accurate blood pressure data of the user of the smart watch, the physiological sound signal collector, the electrocardiogram signal collector, the pulse signal collector and the control chip can be integrated in the body and/or the strap, and the integration position of the above components and chip in the body is not specifically limited in this embodiment.

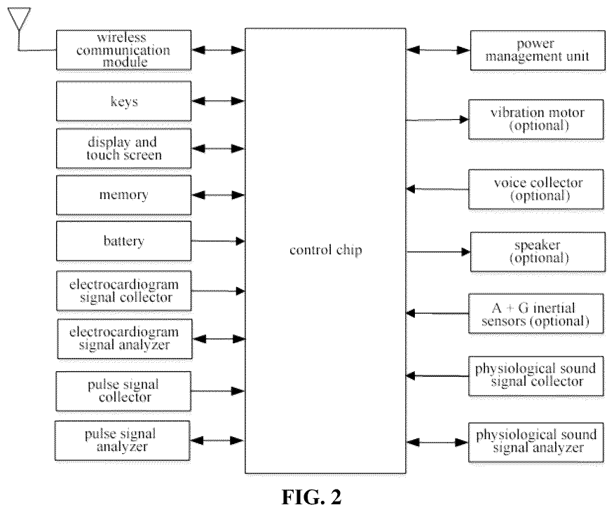

Specifically, as shown in FIG. 2, the physiological sound signal collector, the electrocardiogram signal collector, the pulse signal collector, and the control chip are included in the body of the smart watch. Here, the physiological sound signal collector is configured to collect a physiological sound signal of a user of the smart watch, the electrocardiogram signal collector is configured to collect an electrocardiogram signal of the user of the smart watch, the pulse signal collector is configured to collect a pulse signal of the user of the smart watch, and the control chip is configured to calculate physiological data of the user of the smart watch according to the collected physiological sound signal, the electrocardiogram signal, and/or the pulse signal. Here, each unit is connected to the control chip to transmit respective collected signals to the control chip and is further processed by the control chip.

The physiological sound signal in this embodiment may include a heart sound signal/lung sound signal, and the physiological data includes one or more of blood pressure data and cardiopulmonary disease data of the user of the smart watch.

In an embodiment, the physiological sound signal collector includes one or more of at least one bone conduction sensor, an acceleration sensor, an inertial sensor, a piezoelectric ceramic, a pressure sensor, and a vibration detection sensor; the electrocardiogram signal collector includes at least two electrocardiogram electrodes, and the pulse signal collector includes an LED light source and a detector.

In order to accurately measure the blood pressure of the user of the smart watch, in this embodiment, the physiological sound signal collector is integrated with one or more of at least one bone conduction sensor, an acceleration sensor, A (accelerometer)+G (gyroscope) inertial sensors, a piezoelectric ceramic, a force sensor, and a vibration detection sensor.

For example, when there are two or more bone conduction sensors, a plurality of bone sensors can be arranged in a sensor array.

In addition, the electrocardiogram signal collector includes at least two electrocardiogram electrodes, and the two electrocardiogram electrodes may be ECG_P and ECG_N for measuring the electrocardiogram signal of the user of the smart watch.

The pulse signal collector includes an LED light source and a detector, wherein the detector may specifically be a PD device (photodiode), wherein the LED light source is configured to emit detection light, which may be received by the PD device after being reflected and absorbed by human blood vessels and tissues.

In an embodiment, the physiological sound collector may be installed on a body of the smart watch and/or a strap of the smart watch.

In this embodiment, the physiological sound collector may be mounted on a body and/or a strap of the smart watch. For example, the electronic device may be installed at a position such as a screen, a middle frame, a lower case, or a strap of the smart watch that can effectively collect a physiological sound signal.

Specifically, for example, when the physiological sound collector includes a plurality of sensors (for example, at least one bone conduction sensor, an acceleration sensor, A +G inertial sensors, a piezoelectric ceramic, a pressure sensor, and a vibration detection sensor), the plurality of sensors may be separately installed on the body and the strap, to collect the physiological data of the user of the smart watch from a plurality of dimensions, thereby improving accurate collection of the physiological data.

For another example, when the physiological sound collector includes at least one bone conduction sensor, in order to receive a heart sound signal with higher quality, the at least one bone conduction sensor may be disposed on the lower case of the body of the smart watch and closely contact the lower case, so that the bone conduction sensor can be in close contact with the wrist of the wearer. Similarly, when a plurality of bone conduction sensors form a sensor array, the sensor array may also be mounted in close contact to the lower case.

In an embodiment, the electrocardiogram electrodes include at least a first electrode and a second electrode, wherein the first electrode is mounted on a lower case of the body of the smart watch, and the second electrode is mounted on an upper case or a side wall of the body of the smart watch.

The electrocardiogram electrodes further include a third electrode, and the third electrode is mounted on the lower case of the body of the smart watch.

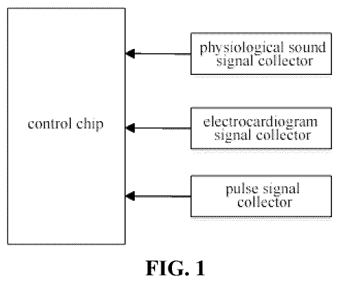

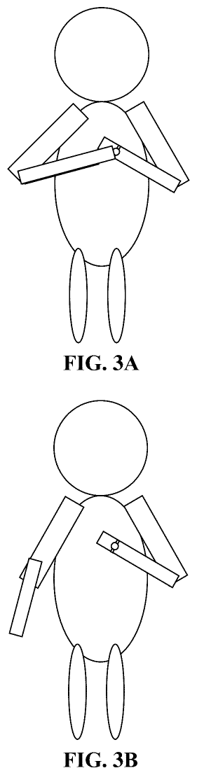

The electrocardiogram signal collector of the smart watch includes at least two electrodes, that is, a first electrode ECG_P and a second electrode ECG_N, where the first electrode ECG_P is mounted on the lower case of the smart watch and closely contacts the lower case, and the second electrode ECG_N is mounted on the upper case or the side wall of the smart watch, so that the user of the smart watch can correctly wear the smart watch on the wrist, and after the smart watch is worn, as shown in FIG. 3-1, the worn smart watch closely contacts the chest, and the finger of the other hand not wearing the smart watch is lightly touched on the second electrode ECG_N located on the upper case or the side wall of the body of the smart watch. Here, the first electrode ECG_P located on the lower case of the smart watch can closely contact the user's wrist to constitute a circuit path. This ensures the quality of the collected electrocardiogram signals, thereby improving the accuracy of the measured blood pressure data. During this process, the smartwatch user will not experience any discomfort, greatly enhancing the user experience.

In addition, the third electrode ECG_RLD may also be further mounted on the lower case of the smart watch as a reference electrode, and may be used together with the first electrode ECG_P and the second electrode ECG_N to collect the electrocardiogram signal of the user of the smart watch.

In an embodiment, the LED light source and the detector are mounted on a lower case of a body of the smart watch.

The pulse signal collector of the smart watch includes an LED light source and a PD device, specifically, a transparent cover plate is provided on the lower case of the body of the smart watch, the LED light source (including a red light, an infrared light, a green light, etc.) and the PD device (i.e., a detector) for collecting incident light (attenuated light emitted by the LED light, emitted by the LED light, reflected and absorbed by human blood vessels and tissues, etc.) are provided under the transparent cover plate, and the LED light and the PD device are jointly used for measuring a pulse signal for the smart watch.

In an embodiment, the smart watch further includes:
a physiological sound signal analyzer configured to analyze and process the physiological sound signal collected by the physiological sound signal collector, and transmit the analyzed and processed physiological sound signal to the control chip;
an electrocardiogram signal analyzer configured to analyze and process the electrocardiogram signal collected by the electrocardiogram signal collector, and transmit the analyzed and processed electrocardiogram signal to the control chip; and
a pulse signal analyzer configured to analyze and process the pulse signal collected by the pulse signal collector, and transmit the analyzed and processed pulse signal to the control chip.

As shown in FIG. 2, in order to analyze the physiological sound signal, the electrocardiogram signal and the pulse signal collected by the collector, a physiological sound signal analyzer, an electrocardiogram signal analyzer and a pulse signal analyzer are further integrated of the smart watch.

Specifically, for example, after collecting the physiological sound signal, the electrocardiogram signal and the pulse signal of the user of the smart watch, the smart watch transmits the physiological sound signal to the physiological sound signal analyzer of the smart watch through the control chip, and transmits the electrocardiogram signal to the electrocardiogram signal collector of the smart watch and transmits the pulse signal to the pulse signal analyzer of the smart watch, and the physiological sound signal analyzer, the electrocardiogram signal analyzer and the pulse signal analyzer analyze and process the collected signals respectively, and return the analyzed and processed signals to the control chip, and the control chip performs calculation on the analyzed and processed signals to collect the physiological data of the user of the smart watch.

In an embodiment, the smart watch further includes:
a speaker, a displayer and/or a vibration motor configured to remind the user of the smart watch to wear the smart watch on the user's wrist, so that one electrocardiogram electrode of the electrocardiogram signal collector closely contact the wrist of the user of the smart watch, and prompt the user of the smart watch to closely attach the worn smart watch to the user's chest while using the other hand not wearing the smart watch to lightly touch the other electrocardiogram electrode of the electrocardiogram signal collector located on the top or side wall of the body.

As shown in FIG. 2, in addition to the signal collector and the corresponding signal analyzer, at least one of the following components is included in this embodiment: a displayer, a vibration motor, and a speaker, where the displayer may be specifically a display and touch screen on the smart watch, and is configured to perform corresponding text display and picture or video display for the user of the smart watch; and the speaker may include a microphone, a speaker, and the like, and the vibration motor may include a vibration motor, to drive the smart watch to vibrate.

It may be understood that, in this embodiment, the electrode that is in the electrocardiogram signal collector and that is located on the lower case of the smart watch and that closely contacts the wrist of the user of the smart watch may be the first electrode, or may be the first electrode or the third electrode, and does not affect measurement of the electrocardiogram signal of the user of the smart watch, which is not specifically limited in this embodiment.

Specifically, as shown in FIG. 3-1, in order to improve the accuracy of the measured physiological data and ensure the quality of the measured physiological data, the smart watch can detect whether it is currently in the physiological data measurement function state in real time. Once the smart watch detects that it is currently in the physiological data measurement function state, in order to detect the physiological sound signal, the electrocardiogram signal and the pulse signal with high quality, the smart watch reminds the user of the smart watch to correctly wear the smart watch on the user's wrist through one or more of the above speaker, the displayer and the vibration motor, so that the first electrode (or the first electrode and the third electrode) of the electrocardiogram signal collector closely contacts the wrist of the user of the smart watch, and prompt the user of the smart watch to tightly attach the worn smart watch to the user's chest, while using the other hand not wearing the smart watch to lightly touch the second electrode of the electrocardiogram signal collector located on the top or side wall of the body.

In another embodiment, as shown in FIG. 3-2, in a state in which the smart watch is normally worn on the user's wrist, the user may lift the wrist to closely contact the chest, to perform health measurement, and does not have to remove the smart watch device to closely contact the chest to perform health measurement.

Therefore, the plurality of physiological data measurement manners of the smart watch in this embodiment can improve convenience and use experience of user measurement.

In an embodiment, the control chip is further configured to determine whether the signal qualities of the physiological sound signal, the electrocardiogram signal and the pulse signal are greater than corresponding preset quality thresholds, so as to determine the physiological data of the user of the smart watch when the signal qualities are respectively greater than the corresponding preset quality thresholds.

In this embodiment, before the control chip processes the physiological sound signal, the electrocardiogram signal, and the pulse signal collected by each collector to collect the blood pressure data, in order to avoid that the measured physiological data cannot accurately reflect the health status of the user because invalid signal data are collected, the control chip may be further configured to pre-determine whether the signal qualities of the collected physiological sound signal, electrocardiogram signal, and pulse signal meet the corresponding preset quality thresholds, so as to calculate the blood pressure data of the user of the smart watch only when it is determined that each signal quality is greater than the corresponding preset quality threshold. Here, each signal has the corresponding preset quality threshold. The preset quality threshold is not specifically limited in this embodiment, and may be flexibly adjusted according to an actual scenario.

In addition, a wireless communication module (including one or a combination of wireless communication such as cellular communication, Wi-Fi communication, and Bluetooth communication, wherein when the smart watch works, a communication connection to another device such as a mobile device may be established, to transmit collected data to a user of the mobile device), a memory, a battery, a power management unit, and a motion sensor (including an accelerometer, a gyroscope, and the like) are further integrated of the smart watch.

Compared with the conventional digital cuff-type pressure monitor or PPG combined with ECG blood pressure measurement method in the related art, the smart watch in the present disclosure has a small structure size, and the measurement method is simple and easy to use. In the present disclosure, the physiological sound signal collector, the electrocardiogram signal collector and the pulse signal collector of the smart watch simultaneously collect the physiological sound signal, the electrocardiogram signal and the pulse signal of the user of the smart watch, and then calculate the physiological data such as the blood pressure data and the cardiopulmonary disease of the user of the smart watch through the control chip of the smart watch by using the physiological sound signal, the electrocardiogram signal and the pulse signal. Therefore, the smart watch in the present disclosure integrates various health data detection functions such as blood pressure measurement and cardiopulmonary disease detection, thereby realizing comprehensive and accurate detection of physiological data of a user of the smart watch. Moreover, the physiological sound measurement method in the present disclosure is simple and convenient to operate, and improves the user experience when measuring physiological data.

On this basis, the present disclosure avoids the disadvantage of squeezing the wrist of the user when the blood pressure is measured by the digital cuff-type pressure monitor, and also solves the problem of inaccurate blood pressure value caused by blood pressure measurement by PPG in combination with ECG blood pressure measurement. Furthermore, in the present disclosure, the blood pressure measurement is performed by using the physiological sound signal, the electrocardiogram signal, and the pulse signal, thereby improving accuracy of blood pressure data measurement, and implementing accurate and efficient blood pressure measurement.

Based on the above hardware structure of the smart watch, a physiological data measurement method applied to the smart watch is proposed, the present disclosure provides an embodiment of the physiological data measurement method, and it should be noted that although a logical order is shown in the flowchart, in some cases, the steps shown or described may be performed in an order different from the order herein.

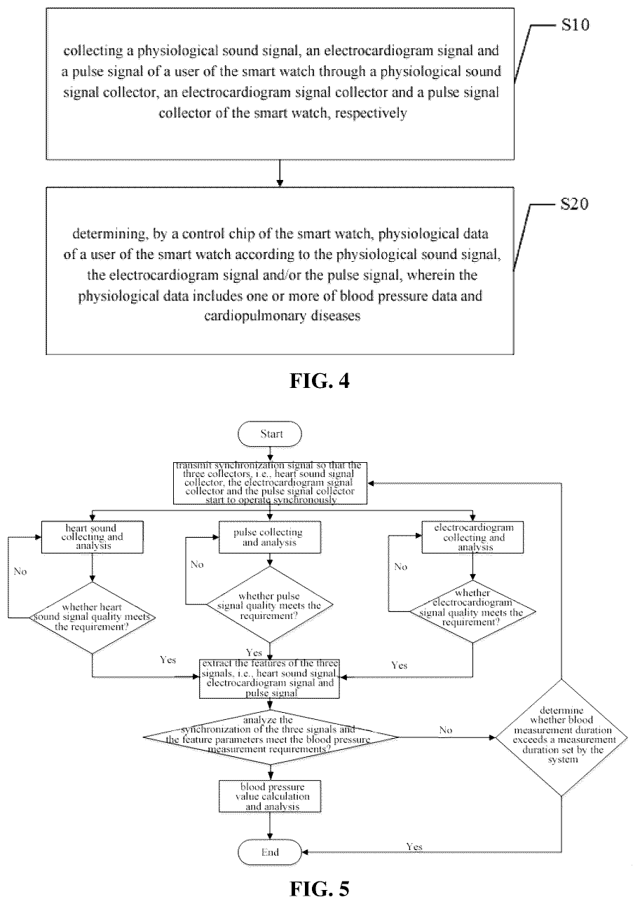

As shown in FIG. 4, the physiological data measurement method in this embodiment specifically includes the following steps:

Step S10, collecting a physiological sound signal, an electrocardiogram signal and a pulse signal of a user of the smart watch through a physiological sound signal collector, an electrocardiogram signal collector and a pulse signal collector of the smart watch, respectively;

After the user correctly wears the smart watch, the smart watch respectively collects the physiological sound signal, the electrocardiogram signal and the pulse signal of the user of the smart watch through the physiological sound signal collector, the electrocardiogram signal collector and the pulse signal collector, so as to further transmit the collected physiological sound signal, electrocardiogram signal and pulse signal to the corresponding physiological sound signal analyzer, electrocardiogram signal analyzer and pulse signal analyzer for analysis and processing, and then transmit the analyzed and processed signals to the control chip of the smart watch for physiological data calculation.

It should be noted that, in this embodiment, in order to ensure that the smart watch can measure a more accurate blood pressure value, the user should wear the smart watch correctly. For example, as shown in FIG. 3, the correct wearing posture of the smart arm is specifically as follows: the user can wear the smart watch on the wrist (the tightness thereof should be suitable, so that the body of the smart watch can closely contact the wrist of the wearer), and further, when blood pressure measurement is performed, the smart watch at the wrist closely contacts the chest, and the other finger not wearing the smart watch is lightly touched and pressed against the ECG electrode on the upper case or the side surface of the smart watch, so that the ECG electrode close to the arm of the wearer on the lower case of the smart watch and the ECG electrode close to the worn finger on the upper case or the side surface of the smart watch form a single-lead configuration, which is used to measure an electrocardiogram signal (i.e., Electrocardiogramave) of the wearer.

Step S20, determining, by a control chip of the smart watch, physiological data of a user of the smart watch according to the physiological sound signal, the electrocardiogram signal and/or the pulse signal, wherein the physiological data includes one or more of blood pressure data and cardiopulmonary disease data.

After collecting the physiological sound signal, the electrocardiogram signal and the pulse signal of the user of the smart watch, the smart watch transmits the above heart sound signals to the physiological sound signal analyzer, the electrocardiogram signal analyzer and the pulse signal analyzer of the smart watch, and the physiological sound signal analyzer, the electrocardiogram signal analyzer and the pulse signal analyzer analyze and process the collected signals respectively, and transmit the analyzed and processed signals to the control chip, and the control chip calculates the corresponding physiological data according to the analyzed and processed signals.

Specifically, for example, when the physiological data in this embodiment is blood pressure data, the control chip can calculate the pulse transmission time and the heart rate value according to the physiological sound signal (the physiological sound signal at this time is a heart sound signal), the electrocardiogram signal and the pulse signal, and finally collect the blood pressure data of the user of the smart watch, such as blood pressure (BP), in combination with the blood pressure calculation regression model, thereby diagnosing the user's physical health status based on the BP value.

Further, when the physiological sound signal includes a heart sound signal and the physiological data includes blood pressure data, the above step S20 of determining, by a control chip of the smart watch, physiological data of the user of the smart watch according to the physiological sound signal, the electrocardiogram signal and the pulse signal may include:

Step S201: determining, by the control chip of the smart watch, the blood pressure data of the user of the smart watch according to the heart sound signal, the electrocardiogram signal, and the pulse signal.

In this embodiment, when the physiological sound signal includes a heart sound signal, and correspondingly, the physiological data is blood pressure data, the control chip can calculate the blood pressure data of the user of the smart watch according to the heart sound signal, the electrocardiogram signal, and the pulse signal.

Specifically, after the smart watch collects the heart sound signal, the electrocardiogram signal and the pulse signal, in order to further calculate the blood pressure of the user of the smart watch, the smart watch may pre-evaluate whether each signal meets the preset blood pressure measurement standard, the preset blood pressure measurement standard in this embodiment may specifically be: the signal features of each signal have synchronization (that is, whether the change rule between the signals has a linear relationship), for example, the R wave of the electrocardiogram signal corresponding to the same heartbeat of the user of the smart watch generally appear earliest, then the heart sound signal, and finally the pulse signal, and if the collected heart sound signal, electrocardiogram signal and pulse signal are not in this order, or the delays between the three signals are significantly greater than the preset regular value (the abnormal situation of the individual is considered in the preset regular value), the collected three signals should not be used for blood pressure calculation and may be discarded. Further, when it is determined that each signal feature meets the preset blood pressure measurement standard, the blood pressure of the user of the smart watch is calculated according to the collected heart sound signal, electrocardiogram signal and pulse signal.

In an embodiment, a specific manner of determining whether each signal feature meets the preset blood pressure measurement standard may be: After collecting the heart sound signal, the electrocardiogram signal, and the pulse signal, the smart watch separately extracts signal features corresponding to the heart sound signal, the electrocardiogram signal, and the pulse signal, and then collects a coherence coefficient between the signal features, and determines, based on the correlation coefficient, whether each signal feature has synchronization, to determine whether each signal feature meets the preset blood pressure measurement standard.

In another embodiment, as shown in FIG. 5, if the smart watch detects that at least one of the signal feature of the heart sound signal, the signal feature of the electrocardiogram signal, and the signal feature of the pulse signal does not meet the foregoing preset blood pressure measurement standard, it means that an invalid signal exists, and in order to avoid a big error in the blood pressure measurement value caused by performing blood pressure calculation by using the invalid signal data, the smart watch may be further configured to collect whether the current blood pressure measurement duration exceeds the preset measurement duration. The current blood pressure measurement duration herein refers to a duration in which the smart watch is currently in a blood pressure measurement function state, and the preset measurement duration refers to a duration commonly required by most users to perform blood pressure measurement by using the smart watch. Further, when the smart watch detects that the current blood pressure measurement duration exceeds the preset measurement duration, to improve blood pressure measurement efficiency, the smart watch directly ends this blood pressure measurement operation. On this basis, the user of the smart watch may be further reminded that this blood pressure measurement operation fails, and a next blood pressure measurement operation is performed in time. If it is detected that the current blood pressure measurement duration does not exceed the preset measurement duration, the heart sound signal may be collected again by the physiological sound signal collector, and the electrocardiogram signal and the pulse signal may be collected by the PPG +ECG signal collector to calculate the user's blood pressure again. It can be learned that, in this embodiment, after an invalid signal is collected, whether to end this blood pressure measurement operation or continue to re-collect a signal can be determined based on current blood pressure measurement duration, so that interference of the invalid signal on blood pressure calculation is eliminated, blood pressure measurement efficiency is greatly improved, and blood pressure measurement precision is improved. On this basis, reliable data is collected for subsequent user's health status monitoring.

Further, before the step S201 of determining blood pressure data of the user of the smart watch according to the heart sound signal, the electrocardiogram signal, and the pulse signal, the method further includes:

Step S202: determining whether signal qualities of the heart sound signal, the electrocardiogram signal, and the pulse signal meet corresponding preset quality thresholds, respectively;

Step S203, if the signal qualities of the heart sound signal, the electrocardiogram signal and the pulse signal are greater than the preset quality thresholds, respectively, performing the step of determining the blood pressure data of the user of the smart watch according to the heart sound signal, the electrocardiogram signal and the pulse signal.

After the smart watch collect the heart sound signal, the electrocardiogram signal and the pulse signal of the user of the smart watch, and before the above signals are processed to collect the blood pressure data, in order to avoid that the measured blood pressure cannot accurately reflect the health status of the user due to the collected invalid signals, as shown in FIG. 5, the smart watch may pre-determine whether the signal quality of the collected heart sound signal, electrocardiogram signal and pulse signal meet the corresponding preset quality thresholds. Here, each signal has the corresponding preset quality threshold, which is not specifically limited in this embodiment, and can be flexibly adjusted according to actual scenarios.

Further, if the smart watch determines that the signal qualities of the collected heart sound signal, electrocardiogram signal, and pulse signal are greater than the corresponding preset quality thresholds, the smart watch can perform blood pressure calculation according to the heart sound signal, cardiac electrical signal, and pulse signal.

Further, when the physiological sound signal includes a heart sound/lung sound signal, the above step S20 may further include:

Step S204, determining, by the control chip of the smart watch, the cardiopulmonary disease data of the user of the smart watch according to the heart sound/lung sound signal. Here, the cardiopulmonary disease data includes one or more of heart failure, coronary heart disease, valvular heart disease, pneumonia, and crackles.

It can be understood that, for crackle-related cardiopulmonary disease data, sound is generated when the airflow passes through the bronchi with thin secretions, liquid vibration or bubble rupture. The sound is characterized by being intermittent and transient, and is mostly found in the inhalation phase. Specifically, crackles are divided into coarse crackles, medium crackles, fine crackles (also known as large, medium, and small bubble sounds) or rales. And it is mainly found in bronchial diseases (such as chronic obstructive pulmonary disease and bronchiectasis), infectious or non-infectious pulmonary inflammation, pulmonary edema and alveolar hemorrhage. Different types of crackles indicate that the main parts of thin secretions are different. For example, fine crackles often occur during pneumonia, and coarse, medium and fine crackles can occur at the same time during acute pulmonary edema.

Heart failures are classified into left heart failure, right heart failure, and global heart failure. In case of left heart failure, cardiac auscultation typically reveals cardiomegaly and a regurgitant murmur caused by relative mitral valve insufficiency, in addition to the inherent signs of the patient's underlying heart disease. Other findings may include accentuation of the second heart sound (S2) in the pulmonary valve area, as well as a third or fourth heart sound gallop (S3 or S4 gallop rhythm). In right heart failure, cardiac auscultation may detect a significantly enlarged right ventricle, leading to a regurgitant murmur due to tricuspid valve insufficiency. In global heart failure, both the left and right heart are enlarged, which may result in relative mitral and tricuspid valve insufficiency with regurgitant murmurs. In the physical examination of heart failure, there may be no distinct clinical manifestations, and cardiac auscultation serves only as a supplementary reference.

According to the features of cardiopulmonary disease described above, in this embodiment, the cardiopulmonary disease of the user of the smart watch may be detected by using the heart sound/lung sound collected by the physiological sound collector, where the physiological sound collector in this embodiment may be a heart sound/lung sound collector.

It should be noted that, in this embodiment, according to the above description, the heart sound/lung sound collector includes at least one bone conduction sensor, acceleration sensor, A+G inertial sensors, piezoelectric ceramic, force sensor (pressure sensor), or vibration detection sensor, etc., and these sensors are fixed on the smart watch, such as the body or strap of the smart watch. The position of the sensor is not specifically limited in this embodiment, as long as the heart sound and lung sound signals can be effectively collected.

On this basis, the heart sound/lung sound collector collects a signal of heart beating and/or a signal of lung breathing of the user of the smart watch, and converts the signal into an electrical signal. After collecting the electrical signal, the control chip calculates the cardiopulmonary disease data of the user of the smart watch according to the electrical signal.

In another embodiment, the control chip may store the above cardiopulmonary disease data in the storage medium of the smart watch, and at the same time, may transmit the cardiopulmonary disease data to the mobile phone APP or the cloud data center connected to the smart watch through the wireless transmission module of the smart watch, so that the professional doctor may collect the heart sound/lung sound collected by the smart watch through the data stored in the mobile phone APP or the cloud data center, and perform diagnosis and recommendation related to the cardiopulmonary disease according to the collecting position of the heart sound/lung sound.

In an embodiment, after the step S10 of collecting the heart sound signal, the electrocardiogram signal and the pulse signal of the user of the smart watch through the physiological sound signal collector, the electrocardiogram signal collector and the pulse signal collector of the smart watch, respectively, the method may further include:
when the smart watch has not collected the heart sound signal, determining whether a current heart sound signal collecting duration exceeds a preset heart sound collecting duration; and
if the current heart sound signal collecting duration exceeds the preset heart sound collecting duration, triggering a wearing posture adjustment instruction for the smart watch, and prompting the user of the smart watch to adjust a wearing position and/or a heart sound signal collecting position of the smart watch through the wearing posture adjustment instruction.

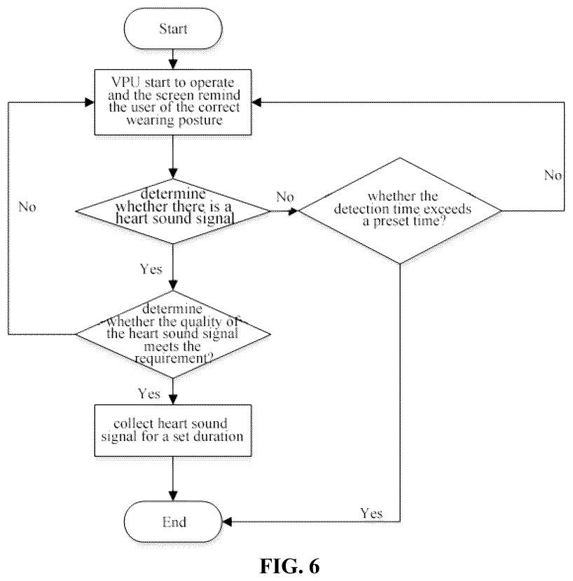

It should be noted that, in this embodiment, as shown in FIG. 6, considering that when a user wears the smart watch to measure blood pressure, the worn smart watch should closely contact a chest, and therefore, there is a problem that the user does not place the smart watch at a correct heart sound signal collecting position or a body of the smart watch does not closely contact a wrist of the user, and the like. It should be noted that, if the smart watch can contact the heart sound signal, it means that the smart watch is worn correctly and the user operates correctly, and in this case, it can be also ensured that the electrocardiogram signal and the pulse signal are collected.

Specifically, for example, when the smart watch cannot collect the heart sound signal, the smart watch may further determine whether the current heart sound signal collecting duration exceeds the preset heart sound collecting duration, wherein the current heart sound signal collecting duration refers to the duration of the smart watch currently in the heart sound collecting state, and the preset heart sound collecting duration refers to the duration generally required by the smart watch to collect the heart sound signal, which is not specifically limited in this embodiment. If it is determined that the current collecting duration of the heart sound signal does not exceed the preset collecting duration of the heart sound signal, the wearing posture adjustment instruction is triggered through the speaker or the displayer of the smart watch to remind the user of the smart watch to adjust the wearing posture of the smart watch and/or the collecting position of the heart sound signal in time.

In another embodiment, if the current heart sound signal collecting duration exceeds the preset heart sound collecting duration, in order to remind the blood pressure measurement efficiency, the smart watch directly ends the current blood pressure measurement operation, and triggers a blood pressure measurement failure reminder, where the blood pressure measurement failure reminder includes a failure reason, for example, the heart sound signal is not collected, to remind the user to correctly perform the next blood pressure measurement operation.

Therefore, in the present disclosure, the smart watch performs blood pressure measurement and cardiopulmonary disease measurement according to the physiological sound signal, the electrocardiogram signal and the pulse signal, which improves the accuracy of physiological data measurement and realizes accurate and efficient blood pressure measurement and cardiopulmonary disease measurement. Moreover, the present disclosure can trigger the wearing reminder of the smart watch, guide the user to correctly wear the smart watch and perform physiological data measurement with a correct operation, ensure the quality of the measured physiological data, simplify the physiological measurement operation, and improve the user's experience in measuring blood pressure and cardiopulmonary disease data. In addition, in order to avoid that the measured physiological data cannot accurately reflect the health status of the user due to the collecting of invalid signals, the present disclosure determines in advance whether the signal qualities of the collected physiological sound signal, electrocardiogram signal and pulse signal meets the corresponding preset quality thresholds, which greatly improves the quality of the collected signal, thereby improving the accuracy of the measured physiological data. In addition, in the present disclosure, it is avoided that there is a great error in the blood pressure measurement value due to the use of invalid signals for blood pressure calculation, and after invalid signals are collected, it can be determined whether to end this blood pressure measurement operation or continue to re-collect signals according to the current blood pressure measurement duration, which eliminates the interference of invalid signals on the calculation of physiological data, greatly improves the physiological data measurement efficiency, improves the detection accuracy of blood pressure and cardiopulmonary disease, and provides reliable data for subsequent user health status monitoring. At the same time, the problem of large blood pressure and physiological sound measurement errors caused by the user failed to place the smart watch at the correct position for physiological sound signal collecting or the body of the smart watch not contact with the user's wrist is solved.

In addition, an embodiment of the present disclosure further provides a computer-readable storage medium, the storage medium stores a blood pressure measurement program, and when the blood pressure measurement program is executed by a processor, the following steps of the physiological data measurement method are implemented.

For each embodiment of the blood pressure measurement device and the computer-readable storage medium of the present disclosure, reference may be made to each embodiment of the physiological data measurement method of the present disclosure, and details are not described herein again.

It should be noted that, in this specification, the terms “include”, “comprise”, or any other variant thereof are intended to cover non-exclusive inclusion, so that a process, method, article, or system that includes a series of elements includes not only those elements, but also other elements that are not explicitly listed, or elements inherent to such a process, method, article, or system. An element proceeded by “comprises a . . . ” does not, without more constraints, preclude the existence of additional identical elements in the process, method, article, or system that comprises the element.

The sequence numbers of the embodiments of the present disclosure are merely for description, and do not represent the preference of the embodiments.

Through the description of the foregoing implementations, a person skilled in the art may clearly understand that the methods in the foregoing embodiments may be implemented by using software and a necessary general hardware platform, or may be implemented by using hardware. Based on such understanding, the technical solutions of the present disclosure essentially or the part that contributes to the prior art may be embodied in the form of a software product, and the computer software product is stored in a storage medium (such as a ROM/RAM, a magnetic disk, or an optical disk), and includes several instructions for enabling a terminal device (which may be a wearable device, a locator, a smartphone, a tablet computer, a server, or another network device) to execute the methods described in the embodiments of the present disclosure.

The above are only preferred embodiments of the present disclosure, and are not intended to limit the patent scope of the present disclosure, and any equivalent structure or equivalent process transformation made by using the content of the specification and the accompanying drawings of the present disclosure, or directly or indirectly applied to other related technical fields, are similarly included in the patent protection scope of the present disclosure.

1. A smart watch comprising:
a physiological sound signal collector configured to collect a physiological sound signal;
an electrocardiogram signal collector configured to collect an electrocardiogram signal;
a pulse signal collector configured to collect a pulse signal; and
a control chip configured to determine physiological data of a user of the smart watch according to the physiological sound signal, the electrocardiogram signal and/or the pulse signal, wherein the physiological data comprises one or more selected from a group consisting of blood pressure data and cardiopulmonary disease data.

2. The smart watch according to claim 1, wherein the physiological sound signal collector comprises one or more selected from a group consisting of at least one bone conduction sensor, an acceleration sensor, an inertial sensor, a piezoelectric ceramic, a pressure sensor and a vibration detection sensor,
wherein the electrocardiogram signal collector comprises at least two electrocardiogram electrodes, and
wherein the pulse signal collector comprises an LED light source and a detector.

3. The smart watch according to claim 2, wherein the physiological sound collector is installed on a body of the smart watch and/or a strap of the smart watch.

4. The smart watch according to claim 2, wherein the electrocardiogram electrodes comprise at least a first electrode and a second electrode, wherein the first electrode is mounted on a lower case of a body of the smart watch, and the second electrode is mounted on an upper case or a side wall of the body of the smart watch.

5. The smart watch according to claim 4, wherein the electrocardiogram electrodes further comprise a third electrode, and the third electrode is mounted on the lower case of the body of the smart watch.

6. The smart watch according to claim 2, wherein the LED light source and the detector are mounted on a lower case of a body of the smart watch.

7. The smart watch according to claim 1, further comprising:
a physiological sound signal analyzer configured to analyze and process the physiological sound signal collected by the physiological sound signal collector, and transmit the analyzed and processed physiological sound signal to the control chip;
an electrocardiogram signal analyzer configured to analyze and process the electrocardiogram signal collected by the electrocardiogram signal collector, and transmit the analyzed and processed electrocardiogram signal to the control chip;
a pulse signal analyzer configured to analyze and process the pulse signal collected by the pulse signal collector, and transmit the analyzed and processed pulse signal to the control chip.

8. The smart watch according to claim 1, further comprising:
a speaker, a displayer and/or a vibration motor configured to remind the user of the smart watch to wear the smart watch on the user's wrist, so that an electrocardiogram electrode of the electrocardiogram signal collector located on a lower case of the smart watch is in close contact with the wrist of the user of the smart watch, and prompt the user of the smart watch to closely attach the worn smart watch to the user's chest while using the other hand not wearing the smart watch to lightly touch another electrocardiogram electrode of the electrocardiogram signal collector located on a top or side wall of the body of the smart watch.

9. The smart watch according to claim 1, wherein the control chip is further configured to determine whether signal qualities of the physiological sound signal, the electrocardiogram signal and the pulse signal are greater than corresponding preset quality thresholds, so as to determine the physiological data of the user of the smart watch when each of the signal qualities is greater than the corresponding preset quality thresholds.

10. A physiological data measurement method applied to the smart watch according to claim 1, comprising:
collecting a physiological sound signal, an electrocardiogram signal and a pulse signal of a user of the smart watch by a physiological sound signal collector, an electrocardiogram signal collector and a pulse signal collector of the smart watch, respectively; and
determining, by a control chip of the smart watch, physiological data of the user of the smart watch according to the physiological sound signal, the electrocardiogram signal and/or the pulse signal,
wherein the physiological data comprises one or more selected from a group consisting of blood pressure data and cardiopulmonary disease data.

11. The physiological data measurement method according to claim 10, wherein the physiological sound signal comprises a heart sound signal, and the determining the physiological data of the user of the smart watch according to the physiological sound signal, the electrocardiogram signal and the pulse signal comprises:
determining, by the control chip of the smart watch, the blood pressure data of the user of the smart watch according to the heart sound signal, the electrocardiogram signal, and the pulse signal.

12. The physiological data measurement method according to claim 11, wherein before the determining the blood pressure data of the user of the smart watch according to the heart sound signal, the electrocardiogram signal and the pulse signal, the method further comprises:
determining whether signal qualities of the heart sound signal, the electrocardiogram signal and the pulse signal are greater than preset quality thresholds, respectively;
if the signal qualities of the heart sound signal, the electrocardiogram signal and the pulse signal are greater than the preset quality thresholds, respectively, performing the determining the blood pressure data of the user of the smart watch according to the heart sound signal, the electrocardiogram signal and the pulse signal.

13. The physiological data measurement method according to claim 10, wherein the physiological sound signal comprises a heart sound/lung sound signal, the determining, by the control chip of the smart watch, the physiological data of the user of the smart watch according to the physiological sound signal comprises:
determining, by the control chip of the smart watch, the cardiopulmonary disease data of the user of the smart watch according to the heart sound/lung sound signal, wherein the cardiopulmonary disease data comprises one or more selected from a group consisting of heart failure, coronary heart disease, valvular heart disease, pneumonia, and crackles.
